(19) United States
(12) Patent Application Publication
(10) Pub. No.: US 2009/0305244 A1
(43) Pub. Date: Dec. 10, 2009

(54) Selection, Propagation and Use of Mosaic Aneuploid Stem Cells

(75) Inventors: Suzanne Earlene Peterson, San Diego, CA (US); Yun Chun Yung, La Jolla, CA (US); Stevens Kastrup Rehen, Rio de Janeiro (BR); Jurjen Willem Westra, San Diego, CA (US); Jerold Jun Chun, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 12/084,750

(22) PCT Filed: Nov. 9, 2006
(86) PCT No.: PCT/US06/43794

§ 371 Date: Feb. 26, 2009

Related U.S. Application Data
(60) Provisional application No. 60/735,715, filed on Nov. 9, 2005.

(51) Int. Cl. C12Q 1/68; C12Q 1/02

(52) U.S. Cl. 435/6; 435/29

(57) ABSTRACT

The distribution of cell karyotypes within a population of cells can determine the phenotype and ability of stem cells to differentiate into desired cell types, to function normally, as well as represent risk for adverse events like cancer. Therefore, determination of the aneuploid mosaic status of a cell population is useful in identifying and/or maintaining desirable traits and eliminating undesirable traits in stem cells, and for defining them at the level of their chromosomal complement.

### CROSS REFERENCE TO RELATED PATENT APPLICATIONS

The present application claims benefit of priority to U.S. Provisional Patent Application No. 60/735,715, filed Nov. 9, 2005, which is incorporated by reference in its entirety.

### STATEMENT AS TO RIGHTS TO INVENTIONS MADE UNDER FEDERALLY SPONSORED RESEARCH AND DEVELOPMENT

This invention was made with Government support under Grant No. K02MH01723, awarded by the National Institutes of Health. The government has certain rights in this invention.

### BACKGROUND OF THE INVENTION

Stem cells have the potential to develop into many different cell types in the body. Stem cells can theoretically divide without limit to replenish other cells. When a stem cell divides, each new cell has the potential to either remain a stem cell or become another type of cell with a more specialized function, such as a muscle cell, a red blood cell, or a brain cell. Stem cells are often classified as totipotent or pluripotent. A totipotent stem cell has differentiation potential which is total: it gives rise to all the different types of cells in the body. A fertilized egg cell is an example of a totipotent stem cell. Pluripotent stem cells can give rise to any cell type in the body derived from the three main germ cell layers or an embryo itself. Progenitor cells can also differentiate into specialized cells. However, in contrast to stem cells, progenitor cells are unable to self-renew and they give rise to only one or a few cell types.

Stem cells include embryonic stem cells and adult stem cells. Embryonic stem cells are derived from embryos. For research purposes, embryonic stem cells are obtained from embryos that have developed from eggs that have been fertilized in vitro (such as at an in vitro fertilization clinic) and then donated for research purposes with informed consent of the donors. The embryos are typically obtained at four or five days old when they are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 30 cells at one end of the blastocoel.

Embryonic stem cells can be obtained, for example, by isolating the inner cell mass and growing them in vitro. The inner cell mass is usually grown on a layer of feeder cells, such as mouse embryonic fibroblasts, that serve as an adherent layer for the inner cell mass and as a source of nutrients. Embryonic stem cells are pluripotent and can become any cell type in the body.

An adult stem cell, or a somatic stem cell, is an undifferentiated cell. Such cells can often be identified among differentiated cells in a tissue or organ. An adult stem cell can renew itself and can differentiate into specialized cell types of the tissue or organ.

Human embryonic stem cells (HESCs) have great potential for use in both basic science and therapeutic strategies, including transplantation for regenerative medicine (Amit M, et al. Dev Biol 227:271-278 (2000)). A challenge for using HESCs is the maintenance of stable cell lines, particularly following extended passaging (Amit M, et al. Dev Biol 227:271-278 (2000); Carpenter M K, et al. Dev Dyn 229:243-258 (2004); Rosler E S, et al. Dev Dyn 229:259-274 (2004)). Indeed, chromosomal instability of NIH-funded HESC lines such as H1, H7, H9 has recently been reported, resulting from clonal expansion of aneuploid stem cells (Draper J S, et al. Nat Biotechnol 22:53-54 (2004); Lakshmipathy U, et al. Stem Cells 22:531-543 (2004); Pera M F, Nat Biotechnol 22:42-43 (2004)). The most frequently reported chromosomal abnormalities were hyperploidies, particularly trisomies of chromosomes 12, 17 or 20 (Draper J S, et al. Nat Biotechnol 22:53-54 (2004); Lakshmipathy U, et al. Stem Cells 22:531-543 (2004); Pera M F, Nat Biotechnol 22:42-43 (2004)), although the generality of chromosomal instability is uncertain (Thomson J A, et al. Science 282:1145-1147 (1998); Amit M, et al. Dev Biol 227:271-278 (2000); Reubinoff B E, et al. Nat Biotechnol 18:399-404 (2000); Thomson J A, et al. Trends Biotechnol 18:53-57 (2000); Xu C, et al. Nat Biotechnol 19:971-974 (2001); Cheng L, et al. Stem Cells 21:131-142 (2003)).

Previously, cytogeneticists have ignored cytogenetic analyses that did not identify all chromosomes because it was believed that missing chromosomes represented inaccuracies of the cytogenetic analysis rather than a true absence of a chromosome. Indeed, the Association of Genetic Technologist's cytogenetics lab manual instructs:
If fewer than 45 chromosomes are present in the metaphase [spread], it can be assumed that some have become lost in the processing and that the metaphase [spread] is unsuitable for analysis.
   See, Barch et al., (1997) The AGT Cytogenetics Laboratory Manual. Lippincott: Philadelphia.

The present invention addresses the role of aneuploidy in stem cells and provides tools for stem cell use and analysis, among other issues.

### BRIEF SUMMARY OF THE INVENTION

The present invention provides methods for detecting and defining the aneuploid mosaic status of a population of stem or progenitor cells. In some embodiments, the methods comprise detecting the presence of aneuploid mosaicism in the population, wherein at least 3 different karyotypes are detected amongst different cells in the population.

In some embodiments, at least 30 cells in the population are screened for aneuploidy.

In some embodiments, the aneuploidy of the cells is recorded.

In some embodiments, at least 4, 5, 6, 7, 8, 9, 10, 15, 20 or more different karyotypes are detected within different cells in the population.

In some embodiments, the methods comprise detecting cells with a net hypoploid mosaic karyotype, and selecting from the cells with a net hypoploid mosaic karyotype a stem or progenitor cell line that is hypoploid for at least a portion of a chromosome for differentiation, further propagation, or transplantation.

In some embodiments, the methods comprise detecting cells with a net hypoploid mosaic karyotype, and selecting from the cells with a net hypoploid mosaic karyotype a stem or progenitor cell line that is hypoploid for at least a portion of a chromosome for drug screening.

In some embodiments, the methods comprise detecting cells with a net hyperploid mosaic karyotype, and selecting from the cells with a net hyperploid mosaic karyotype a stem or progenitor cell line that is hyperploid for at least a portion of a chromosome for differentiation, further propagation, or transplantation.

In some embodiments, the methods comprise detecting cells with a net hyperploid mosaic karyotype, and selecting from the cells with a net hyperploid mosaic karyotype a stem or progenitor cell line that is hyperploid for at least a portion of a chromosome for drug screening (e.g., screening for a drug that either selectively inhibits or kills the cells).

In some embodiments, the methods further comprise passaging stem cells through at least one (e.g., at least 2, 5, 10, 20, 30, 40, 50, 60 70, 80, 100 or more) cycle of cell division prior to the detecting step.

In some embodiments, the karyotype of at least, e.g., 10%, 20%, 30%, 40%, 50%, 60%, 70%, 80%, 90% of the cells in the cell population is determined.

In some embodiments, the detecting step comprises Fluorescent in situ hybridization (FISH) including multiplex FISH. In some embodiments, the detecting step comprises spectral karyotyping (SKY). In some embodiments, the detecting step comprises G banding. In some embodiments, the detecting step comprises DAPI or other chromosome visualization stains or techniques. In some embodiments, the detecting step comprises flow cytometry.

The present invention also provides for methods of screening for an agent that preferentially inhibits cells incapable of differentiation compared to cells capable of differentiation into a desired cell type. In some embodiments, the methods comprise contacting the agent to cells having a net hyperploid mosaic karyotype, wherein the cells are incapable of differentiation into the desired cell type; and selecting an agent that inhibits propagation of the cells.

In some embodiments, the methods further comprise the steps of: contacting the agent to cells having a hypoploid karyotype, wherein the cells are capable of differentiation into the desired cell type; and selecting an agent that inhibits propagation of the hyperploid cells incapable of differentiation but does not significantly inhibit the propagation of the hypoploid cells capable of differentiation into the desired cell type.

The present invention also provides methods for maintaining or improving a stem cell or progenitor cell population. In some embodiments, the methods comprise: detecting the karyotype of at least one chromosome or a portion thereof in cells in the cell population; and separating cells with a euploid or hypoploid karyotype at the at least one chromosome or portion thereof in the cell population from cells that are hyperploid at the at least one chromosome or portion thereof, thereby maintaining or improving the stem cell or progenitor cell population by removing hyperploid cells while maintaining euploid and hypoploid cells in the cell population.

In some embodiments, the detecting and separating steps are performed fluorescence activated cell sorting (FACS).

In some embodiments, following the separating step, the cells with the euploid or hypoploid karyotype are made to differentiate. In some embodiments, following the separating step, the cells with the euploid or hypoploid karyotype are propagated. In some embodiments, following the separating step, the cells with the euploid or hypoploid karyotype are transplanted into an individual.

### DEFINITIONS

“Aneuploidy” is used in its standard meaning, i.e., any deviation from an exact multiple of the haploid number of chromosomes including gains and/or losses, as well as intrachromosomal alterations. Thus, cells showing deviation from two copies of at least part of the haploid genome, i.e., the presence of more than 2 copies of one or more chromosomes or parts thereof, or absence of one or more chromosomes, or parts thereof would be considered aneuploid. A term that is sometimes used to describe aneuploidy is “aneusomy,” which is encompassed within the present definition of aneuploidy. “Aneuploid mosaicism” as used herein refers to the presence of at least three different karyotypes in the cell population, at least two of which are different aneuploid states. In some embodiments, there will be 4, 5, 6, 7, 8, 9, 10 or more different karyotypes in a cell population

“Drug screening” refers to screening through multiple (e.g., a library of) molecules to identify one or more molecule with a desired biological effect. The molecules, sometimes referred to as “agents,” can include, but are not limited to, small organic molecules or biological molecules, such as antibodies, nucleic acids, peptides, lipids, sugars, or combinations thereof.

A “karyotype” as used herein refers to the number and/or type of chromosomes present in a cell.

A “progenitor cell” refers to a cell that can differentiate into a limited number of cell types, but cannot normally dedifferentiate into stem cells. For example, blood progenitor cells found in bone marrow have, as a part of their natural progression, a commitment to produce terminally differentiated red and white blood cells.

A “stem cell” as used herein refers to a cell that is either totipotent or pluripotent. Totipotent stem cells can differentiate into any cell type, of the body along with the embryonic placenta that supports the developing embryo. Totipotent stem cells can give rise to all cells found in a developing organism, including the placenta. Pluripotent stem cells can develop into many of the three major tissue types: endoderm (e.g., interior gut lining), mesoderm (e.g., muscle, bone, blood), and ectoderm (e.g., epidermal tissues and nervous system), but may show restrictions to their developmental potential (e.g., they may not form placental tissue, or other cell types of a defined lineage).

### BRIEF DESCRIPTION OF THE DRAWINGS

FIG. 1 provides graphs showing the percentage of chromosome counts from early and late passage H7 HESCs. The asterisk represent the expected position of euploid cells (46 chromosomes) in the graph. Note that both lines show a significant proportion of hypoploid cells in the early passage populations, while late passage cells tend to show clonal hyperploidies.

FIG. 2 provides graphs showing the percentage of chromosome counts from early and late passage H9 HESC line. The asterisk represent the expected position of euploid cells (46 chromosomes) in the graph. Note that both lines show a significant proportion of hypoploid cells in the early passage populations, while late passage cells tend to show clonal hyperploidies.

FIGS. 3-4 illustrate the effects of hyperploidy on neuronal differentiation. Mammalian ESCs were grown on PA6 cells to induce neuronal differentiation. Cells were then treated with or without taxol for 6 days to induce aneuploidy. Chromosome counts were determined and parallel dishes were fixed and stained for the neuronal marker tuj1. FIG. 3 shows that taxol treatment of the cells induces aneuploidy, specifically hyperploidy. FIG. 4 demonstrates that taxol induced hyperploidy significantly reduces neuronal differentiation potential.

### DETAILED DESCRIPTION OF THE INVENTION

### I. Introduction

The present invention relates to the surprising discovery that aneuploid mosaicism plays a role in normally occurring and developing stem cells. Stem cell researchers to date have believed that aneuploidy was necessarily an abnormality and thus was to be avoided for use in transplantation or other therapeutic uses. Indeed, current methods of stem cell isolation and maintenance emphasize the essential importance of euploidy. See, e.g., U.S. Pat. No. 6,200,806.

In contrast, the present invention provides methods for detecting aneuploid mosaic karyotypes in stem cell or progenitor cell populations and provides for use and analysis of stem cells or progenitor cells selected for a particular aneuploid mosaic karyotype. Aneuploid mosaicism refers to the distribution of karyotypes within a cell population. As explained further herein aneuploid mosaicism is in fact not abnormal, but instead is a prominent property of normal stem cell populations. However, the exact aneuploid mosaic makeup of the stem cell population affects both the genotype and can affect the phenotype of the cell population, including the ability of the population to retain its ability to differentiate into multiple different cell types. In contrast to what has been described previously for stem cells, the inventors have found that a mosaic of different karyotypes within a stem cell population is the norm, and it affects the cell populations' phenotype including its physiological properties and ability to function optimally as stem cells. Accordingly, the present invention provides for “population karyotyping” whereby the karyotype of a greater number of cells in a population is determined than has been described previously. Moreover, in contrast to what has been described before, results from population karyotyping allows for selection of cell populations with particular aneuploidies (e.g., hypoploid karyotypes), whereas previously cells with hypoploidy were either ignored or actively discarded. Also, the present invention allows for selecting particular aneuploidies (e.g., hyperploid at a certain chromosome and/or euploid or hyoploid at other chromosomes).

Examples of different karyotypes in a cell population include, but are not limited to, at least one cell with a normal complement of chromosomes (e.g., in humans, pairs of 22 non-sex chromosomes called “autosomes” plus two sex chromosomes) present with at least one aneuploid cell. In addition, in an aneuploid mosaic population, there may be a variety of different karyotypes in a single population. For example, a cell population can include some cells that are hypoploid for part or all of a particular chromosome and other cells that are hypoploid for part or all of a different chromosome. In some embodiments, a cell population can include some cells that are hypoploid for part or all of a particular chromosome and other cells that are hyperploid for part or all of a different chromosome. In some embodiments, a cell population can include some cells that are hyperploid for part or all of a particular chromosome and other cells that are hyperploid for part or all of a different chromosome.

In some embodiments, at least some cells in a cell population have a net hypoploid mosaic karyotype. A “net hypoploid mosaic karyotype” refers to a cell population whose cells are predominantly (e.g., greater than 50, 60, 70, 80, 90, 95 or 99%) hypoploid in one or more chromosomes or parts thereof. In some embodiments, a majority of the cells are hypoploid in one particular chromosome. In other embodiments, hypoploid cells in the population are hypoploid in different (e.g., at least 1, 2, 3, 4, 5 or more) chromosomes or parts thereof. In some cases, cells that are hypoploid for certain chromosomes are euploid and/or hyperploid for other chromosomes or portions thereof. It is understood that while the cell population can have a “net” hypoploid karyotype, the population can contain euploid and hyperploid cells, albeit as a minority of cells.

In some embodiments, at least some cells in a cell population have a net hyperploid mosaic karyotype. A “net hyperploid mosaic karyotype” refers to a cell population whose cells are predominantly (e.g., greater than 50, 60, 70, 80, 90, 95 or 99%) hyperploid in one or more chromosomes or parts thereof. In some embodiments, a majority of the cells are hyperploid in one particular chromosome. In other embodiments, hyperploid cells in the population are hyperploid in different (e.g., at least 1, 2, 3, 4, 5 or more) chromosomes or parts thereof. In some cases, cells that are hyperploid for certain chromosomes are euploid and/or hypoploid for other chromosomes or portions thereof. It is understood that while the cell population can have a “net” hyperploid karyotype, the population can contain euploid and hypoploid cells, albeit as a minority of cells.

In some embodiments, the cell population is made up of a mixed population of hypoploid and hyperploid cells and/or cells that contain both hypoploid and hyperploid chromosomes or parts thereof. An example of this last aspect is a cell that has three copies of at least a part of a first chromosome but only one copy of at least a part of a second chromosome. In yet another example, at least some cells in the cell population contain a chromosomal translocation.

The present invention is useful for any type of stem or progenitor cells. Exemplary types of stem cells include embryonic stem cells and adult stem cells. Stem cells can be from any type of animal, including human and non-human mammals. Thus, stem cells used in the present invention include human adult stem cells and human embryonic stem cells. Progenitor cells include all of the many progenitor cell types present in a living organism, including but not limited to lineages of hematopoiesis (blood), nervous system, lymphoid, pancreas, cardiac, lung, muscle, bone, cartilage, connective tissue, cornea, hair, skin, liver, intestine, eye, fat, breast, thyroid, sexual reproduction, etc.

In some embodiments, cancer (or otherwise neoplastic) stem cells are isolated by selection of aneuploidies associated with cancer phenotypes. Such cells are useful targets for drug screening with the goal of identifying drugs that specifically inhibit or kill the cancer stem cells. Alternatively, a stem cell population can be sorted such that karyotypes associated with cancer are excluded, thereby allowing the remaining cells in the population to be used in transplantation, etc.

Techniques for isolating stem cells and progenitor cells are well known and are described in, e.g., Thomson et al., Science 282:1145-(1998); Bodnar, et al., Stem Cells and Development 13:243-(2004); Li et al., Current Biology 8:971-(1998); Schwartz et al., Journal of Neuroscience Research 74:838 (2003).

### II. Detecting Aneuploid Mosaicism

In contrast to previous descriptions of stem cells, the inventors have discovered that there is not necessarily clonal expansion of a specific aneuploidy. Therefore, a larger number of cells are analyzed for their karyotype so that a distribution of karyotypes can be determined. Typically this analysis will involve analysis of the karyotype of more cells than typically analyzed previously by stem cell researchers. Those of skill in the art will appreciate that a significant number of cells should be individually karyotyped within a cell population to establish accurately the distribution of karyotypes (i.e., the “aneuploid mosaic”), one of which can include a euploid karyotype, within the cell population. Therefore, in some embodiments more than 30, 50, 75, 100, 150, 200, 300, 500, 1000 or more cells in a cell population are individually karyotyped to establish an accurate distribution of different karyotypes within the cell population. The number of cells analyzed will determine the limit of detection for the occurrence of a particular karyotype. For example, to detect a karyotype that occurs at a 1% frequency, it is necessary to determine the karyotype of at least 100, and more preferably (e.g., 200, 300, 500 or more individual cells in a cell population). In some embodiments, the methods of the invention comprise detecting the karyotype of a sufficient number of individual cells to detect the presence of an individual karyotype that occurs with a frequency of, e.g., 1%, 0.1%, 0.001%, 0.0001%, 0.00001% or less. In some embodiments, the karyotype of all, or substantially all (e.g., at least 80, 90, 95 or 99%), of the cells in a cell population is determined. In general, the karyotyping of individual cells will comprise determining substantially all of the chromosomes in the cell, rather than merely screening for the presence of one specific chromosome or chromosome portion. However, in other embodiments, the present invention also allows for determination of the number of only some chromosomes of a cell (e.g., 2, 3, 4, 5, or more chromosomes).

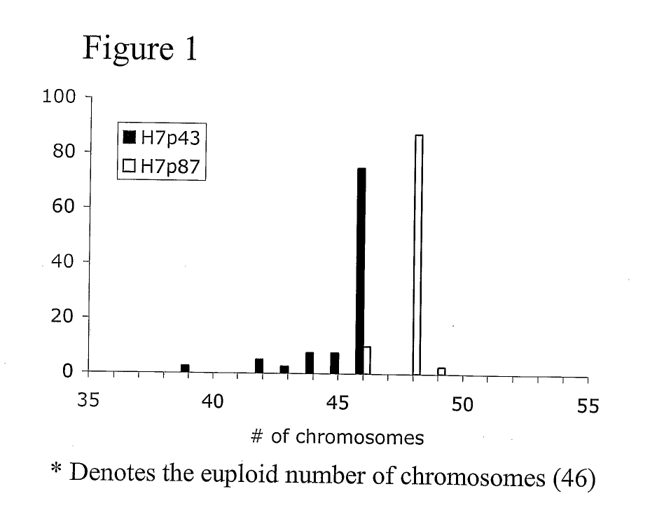

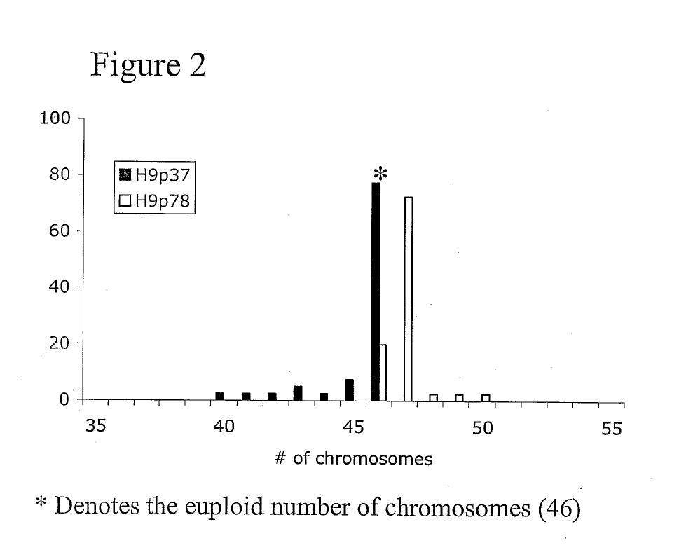

While any type of display may be used to present the data resulting from the detection of aneuploid mosaics, the inventors have found it helpful to display the results in histogram and/or tabular format, thereby visually displaying the quantity of variation of number of different chromosomes or parts thereof within the cell population. An example of such a histogram is displayed in FIGS. 1-2. As FIGS. 1-2 display, the analysis can focus on the total number of chromosomes in each analyzed cell. However, a more detailed analysis of the number of each different chromosome in each cell can also be determined.

Any method available to one of skill in the art can be used to detect aneuploid mosaicism. Karyotyping can be performed to determine chromosome number, chromosome identity and/or chromosome integrity. Chromosome integrity refers to the state of a chromosome as being intact (as would be found normally) or showing evidence of having been disrupted by virtue of breakage, translocation events, micro events that include deletions, translocations, insertions, amplifications, inversions, and any other intra- or inter-chromosomal alteration.

In some embodiments, Fluorescence In Situ Hybridization (FISH) methods are used to determine the karyotype of cells. FISH methods are well known in the art and are described in, e.g., U.S. Patent Publication No. 2005/0214842. Multiplex FISH (M-FISH) methods are particularly useful for karyotyping multiple chromosomes. Exemplary M-FISH methods include, spectral karyotyping (SKY) methods. SKY methods are described in, e.g., Schrock E, et al. Science 273:494 (1996); Speicher M R, et al. Nat Genet. 2:368 (1996); T, Vignon et al. Nat Genet. 15:406, (1997); Macville, M., et al., Hist. Cell. biol. 108 (4-5):299-305 (1997). SKY methods involve use of multiple chromosomal probes labeled with various fluorescent labels to “paint” chromosomes with detectable labels, thereby allowing assessment and identification of an entire chromosome complement.

In some embodiments, flow cytometry such as Fluorescence Activated Cell Sorting (FACS) is used to determine a karyotype for cells in a cell population. For example, DNA dyes (e.g., propidium iodide, ethidium bromide, Hoechst 33342, 33258, DAPI, etc.) can be used to label DNA in cells, and the cells can be then sorted based on the quantity of signal from the dye. This method provides an estimate of overall quantity of chromosomes based on DNA content, but does not provide specific information about what particular chromosome is gained or lost. Nevertheless, flow cytometry using non-specific DNA dyes or other labels are sufficient to distinguish net hypoploid cells from net hyperploid cells. As an alternative to non-specific DNA dyes, labeled markers that are specific for a particular chromosome can also be used. This latter method is most effective when used on cells that are not actively dividing and therefore are in interphase. These probes can be nucleotide-based, or chemically distinct molecules that can still base-pair, such as peptide nucleic acids (that have a peptide backbone), etc.

While not particularly efficient, large scale traditional karyotyping can also be applied assuming a sufficient number of cells can be analyzed as discussed above. Any stain or optical or biophysical technique that detects chromosomes could be used for karyotyping. In some embodiments, the staining of chromosomes with DAPI/other fluorescent stains or brightfield stains is used. Traditional karyotyping can be performed, e.g., on lymphocytes and amniocytes, using labor intensive methods such as Giemsa staining (G banding). In some embodiments, the detecting step comprises whole genome amplification of single cells using PCR, optionally in combination with DNA arrays, and/or combined with single nucleotide polymorphism data and maps. In vivo approaches using fluorescent tags or other methods can also be used to identify the ploidy of living stem cells (e.g., Kaushal et al., J Neurosci. 23:5599-5606 (2003)).

Since genes are expressed from chromosomes, it will be possible to identify some forms of aneuploidy based on the quantitative or qualitative expression of detectable gene products, allowing a surrogate or correlative marker for aneuploidy to be used. This technique can be combined with standard cell-sorting technologies (e.g., FACS) to allow another means of identifying distinct forms of aneuploidy.

In some embodiments, the invention provides for methods of sorting or separating different cells in a cell population (e.g., stem and/or progenitor cells) based on their karyotype, whereby cells with a euploid or hypoploid karyotype are selected away (or otherwise separated) from cells with a hyperploid karyotype. In some embodiments, cells selected for hypoploidy (e.g., loss of at least one chromosome 1 or part of chromosome 1) may also be hyperploid at another chromosome or portion thereof. However, the selection and sorting will be for the particular hypoploidy (e.g., in the above example, chromosome 1). Alternatively, cells with a euploid or hyperploid karyotype are selected away (or otherwise separated) from cells with a hypoploid karyotype.

Such sorting methods can include any sorting method available in the art, including but not limited to, FACS sorting. These sorting methods are useful, for example, for routine quality maintenance and quality control of stem cell and progenitor populations, especially where it is desirable to remove potentially cancerous hyperploid cells.

### III. Uses of Stem Cells Following Selection

The present invention provides for methods of culturing, growing and/or selecting stem cells or progenitor cells or otherwise using stem or progenitor cells, wherein the methods include a step of monitoring and optionally maintaining a particular aneuploid mosaic. Selection of a particular aneuploid mosaic, i.e., a particular distribution of karyotypes within a cell population, allows for their optimal use in all assays, procedures and approaches that use stem cells or progenitor cells. This includes, but is not limited to, stem cells or progenitor cells used for transplantation, producing biological agents (antibodies, siRNAs, etc.), screening for pharmaceutical agents (e.g., molecules that interfere with or enhance stem cell proliferation or differentiation), screening for toxins and/or their effect, detecting bio-defense agents whereby defined mosaics have identified properties that allow agent detection, assessing new agents for cancer treatment, tissue generation, tissue regeneration, vaccination whereby stem or progenitor cells have properties that can allow appropriate antigen presentation or promotion/inhibition of an immune response, genotyping of stem or progenitor cells for both prognostic and diagnostic purposes, including normal and pathological specimens, treatment of genetic disorders, treatment of non-genetic disorders, biosensors, whereby stem or progenitor cells have been previously selected to respond to distinct stimuli that can then respond to internal or external stimuli when introduced into an organism or as a stand-alone monitor, treatment of injury, plastic surgery, and/or production of growth factors, anti-apoptotic or other proteins.

The particular type of aneuploid mosaic selected will depend on the stem or progenitor cell type used. In some embodiments, it is desirable to select and/or maintain a cell population with a net hypoploid mosaic. In some embodiments, it is desirable to select and/or maintain a cell population with a net hyperploid mosaic. In some embodiments, it will be desirable to select and/or maintain a cell population with a particular combination of karyotypes (e.g., translocations and or mixture of cells with different hypoploidy and/or hyperploidy and/or euploidy).

The particular type of aneuploid mosaic desired can be readily determined by monitoring the distribution of karyotypes in a cell population and identifying an association of a desired phenotype or ability with a particular karyotype distribution. An example of this process is described in the Examples. The steps in the Examples include identification of a karyotype distribution of stem cells, identifying the karyotypic difference between at least two different stem cell populations, and then identifying an association of a desired phenotype with one of the karyotype distributions (e.g., stem cells with a net hypoploid distribution retain an ability to differentiate into desired cell types).

Stem cells have gained considerable interest as a treatment for myriad diseases, conditions, and disabilities because they provide a renewable source of cells and tissues. Blood-forming stem cells in bone marrow called hematopoietic stem cells (HSCs) are a commonly used type of stem cell. HSCs are currently used to treat leukemia, lymphoma and several inherited blood disorders. However, HSCs and other stem cells have considerable potential for treating many other diseases. A number of reports have suggested that certain adult stem cell types have the ability to differentiate into multiple cell types. For example, hematopoietic stem cells may differentiate into brain cells (neurons, oligodendrocytes, and astrocytes) (Hao et al., H. Hematother. Stem Cell Res. 12: 23-32 (2003); Zhao et al., Proc. Natl. Acad. Sci. USA 100: 2426-2431 (2003); Bonilla et al., Eur. J. Neurosci. 15: 575-582 (2002)), skeletal muscle cells (Ferrari et al., Science 279: 1528-1530 (1998); Gussoni et al., Nature 401: 390-394 (1999)), cardiac muscle cells (Jackson et al., J. Clin. Invest. 107: 1395-1402 (2001)), and liver cells (Lagasse et al., Nat. Med. 6: 1229-1234, 2000).

Stem cells can be used in the treatment of any kind of organismal problem including, but not limited to, developmental disorders, infections, degenerative disease, physical or chemical injury, including those due to trauma, where tissues need to be replaced or regenerated. Examples of trauma-related conditions include central nervous system (CNS) injuries, including injuries to the brain, spinal cord, or tissue surrounding the CNS injuries to the peripheral nervous system (PNS), or injuries to any other part of the body. Such trauma may be caused by accident, or may be a normal or abnormal outcome of a medical procedure such as surgery or angioplasty. The trauma may be related to a rupture or occlusion of a blood vessel, for example, in stroke or phlebitis. In specific embodiments, the cells may be used in autologous or heterologous tissue replacement or regeneration therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues. Injuries may be due to specific conditions and disorders including, but not limited to, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharide nosis, chronic granulomatous disease and tyrosinemia, Tay-Sachs disease, cancer, tumors or other pathological or neoplastic conditions.

Stem cells can optionally contain an exogenous nucleic acid vector or biological vector in an amount sufficient to direct the expression of a desired gene(s) in a patient. The construction and expression of conventional recombinant nucleic acid vectors is well known in the art and includes those techniques contained in Sambrook et al., Molecular Cloning: A Laboratory Manual, Vols 1-3 (2d ed. 1989), Cold Spring Harbor Laboratory Press. Such nucleic acid vectors may be contained in a biological vector such as viruses and bacteria, preferably in a non-pathogenic or attenuated microorganism, including attenuated viruses, bacteria, parasites, and virus-like particles.

The nucleic acid vector or biological vector may be introduced into the cells by an ex vivo gene therapy protocol, which comprises excising cells or tissues from a patient, introducing the nucleic acid vector or biological vector into the excised cells or tissues, and reimplanting the cells or tissues into the patient (see, for example, Knoell et al., Am. J. Health Syst. Pharm. 55: 899-904 (1998); Raymon et al., Exp. Neurol. 144: 82-91 (1997); Culver et al., Hum. Gene Ther. 1: 399-410 (1990); Kasid et al., Proc. Natl. Acad. Sci. U.S.A. 87: 473-477 (1990)). The nucleic acid vector or biological vector may be introduced into excised cells or tissues by, for example, calcium phosphate-mediated transfection (Wigler et al, Cell 14: 725 (1978); Corsaro and Pearson, Somatic Cell Genetics 7: 603 (1981); Graham and Van der Eb, Virology 52: 456 (1973)). Other techniques for introducing nucleic acid vectors into host cells, such as electroporation (Neumann et al., EMBO J. 1: 841-845 (1982)), may also be used.

The cells of the invention may also be co-administered with other agents, such as other cell types, growth factors, and antibiotics. Other agents may be determined by those of ordinary skill in the art.

Specific types of stem cells can be identified using cell markers specific for a desired type of stem cell. Cell markers may be lineage markers, metabolic markers, communication markers, growth factors, transcription factors, for example. In specific embodiments, specific cell markers are associated with particular desired stem cells. Cell markers may be detected by methods known in the art, such as by immunochemistry or flow cytometry. Flow cytometry allows the rapid measurement of light scatter and fluorescence emission produced by suitably illuminated cells or particles. The cells or particles produce signals when they pass individually through a beam of light. Each particle or cell is measured separately and the output represents cumulative individual cytometric characteristics. Antibodies specific to a cell marker may be labeled with a fluorochrome so that it may be detected by the flow cytometer.

A skilled artisan recognizes how to determine one or more suitable cell markers. In specific embodiments, for human embryonic stem cells, suitable markers include Oct4, TRA-1-60, TRA-1-81, SSEA-4. Exemplary cell markers for hematopoietic stem cells include CD34+, Sca-1+, AA4.1+ and cKit+, and in specific embodiments these markers denote murine hematopoietic stem cells. In alternative embodiments, human hematopoietic stem cells may be CD34+ or CD34−, CD38+, CD38(−), ckit+, Thy 1₁₀, C1FR+, or a combination thereof. Exemplary markers for neural stem cells include nestin, CD133+, BIM1 and Sox2 for example. Exemplary markers for cardiac stem cells include stem cell antigen-1, CD45(−), CD34(−), Sca1+, or a combination thereof, for example. Intestinal stem markers include A33+, cFMS+, c-myb+, CD45(−), or a combination thereof, for example. Skin stem cell markers include keratin 19.

In some embodiments, stem cells are screened for a particular aneuploid mosaic (i.e., karyotype distribution) to confirm that a particular aneuploid mosaic is present (or optionally selected for a particular mosaic) and then transplanted into an animal (e.g., a human or other mammal). By manipulating their culture conditions, the stem cells can be induced to differentiate into desired cell types (e.g., blood cells, neurons, muscle cells, or other cell types), optionally prior to transplantation. This process can ensure that optimal differentiation and functionality are accrued with the introduced stem cells.

In some embodiments, the distribution of karyotypes is determined before, after and/or during propagation of the cells. In these embodiments, the stem cells divide before, after or both before and after the aneuploid mosaic of the cell population is detected. In some embodiments, detection of aneuploid mosaics during and after cell propagation and after storage is used to confirm and/or select cell populations that retain a desired aneuploid mosaic that is associated with a desired phenotype (e.g., the ability to differentiate into a desired cell type). Propagation can include 1, 2, 5, 10, 50 or more cycles of cell division. The level and forms of aneuploid mosaicism can be altered by defined growth conditions that can be optimized for a desired cell type and desired outcome.

As described in the examples, detection of a karyotype distribution within a cell population can allow for identification of cell populations that do or do not retain the ability to differentiate. Those of skill in the art can take advantage of this discovery to identify and/or use different cell populations with different karyotype distributions to screen for molecules that alter the phenotype, inhibit, kill or induce proliferation of one cell population preferentially compared to a second cell population with a different karyotype distribution. Without wishing to limit the invention to specific embodiments, as one example, a first stem cell population that has a net hypoploid mosaic karyotype associated with the ability to differentiate into desired cell types and a second stem cell population that has a net hyperploid mosaic karyotype associated with the inability to differentiate into the desired cell type(s) can be screened against a library of agents (small organic molecules, or biological agents such as antibodies, siRNAs, nucleic acids, peptides, etc.) and agents that inhibit growth of the hyperploid population can be selected. Since stem cells are believed to exist in neoplastic or cancerous populations, this is also a way to identify anti-cancer agents by focusing on the root of cell proliferation, the cancer stem cell. These agents can be further selected to identify those agents that do not significantly inhibit growth of the net hypoploid cell population, thereby identifying an agent that is useful for maintaining cells with the ability to differentiate as desired. In an alternative, different stem cells could be found to retain an ability to differentiate into a desired cell type(s) when having a net hyperploid mosaic karyotype. In some of those embodiments, agents are identified that inhibit net hypoploid cell populations without significantly affecting net hyperploid cell populations.

### Example

Chromosomal instability produced by extended passaging of human embryonic stem cells (HESCs) represents a potential problem for their safe and efficacious therapeutic use. Recently, karyotypic abnormalities detected by classical cytogenetic techniques were reported in HESC lines. To determine the range of abnormalities in current HESCs after extended passaging, we utilized “spectral karyotyping” (SKY) combined with quantification of chromosome gain and loss, on early and late passage H7 and H9 HESCs, as well as murine ESCs. Examination of every HESC or murine ESC in addition to H7 and H9 and including non-NIH HESCs has produced similar results (data not shown). Early passage cells exhibited hypoploidy, contrasting with late passage cells that exhibited primarily clonal hyperploidy. Mosaic aneuploid and euploid mammalian ESC cultures were assessed for their ability to differentiate into neurons, and hyperploid cells showed a significant reduction in differentiation potential. These data identify physiological consequences of mammalian ESC aneuploidy and support routine monitoring of ESCs for distinct forms of aneuploidy.

### Introduction:

The H7 and H9 HESC lines (from WiCell Research Institute, Madison, Wis.,) were analyzed between passage 36-44 (early passage) and passage 77-88 (late passage). SKY was used to examine chromosomal complement and organization, combined with extensive quantification of chromosome gain and loss. Rather than identifying and reporting only representative or conserved forms of aneuploid cells as is currently used for classical cytogenetic approaches, all acceptable metaphase spreads were quantified with respect to the number of lost or gained chromosomes, and these values are plotted graphically. The significance of the observed aneuploidies was examined by assessing the ability of aneuploid mammalian ESCs to differentiate into specific cell types, such as neurons.

Here we report that HESC lines can show a range of aneuploidies that include hyperploidy as well as previously undocumented forms of hypoploidy.

### Results:

### HESCs Show Random Chromosome Gain and Loss

Prior studies using classical cytogenetics have reported that HESCs are essentially euploid at low passages (Thomson et al., 1998; Amit et al., 2000; Draper et al., 2004a; Draper et al., 2004b; Rosler et al., 2004). Recently, Draper and colleagues reported that after 60 passages, H1 subclones H1.1A and H1.1B acquired chromosomal changes characterized specifically by gain of chromosome 12 or 17 (Draper et al., 2004b). To determine the conservation of this aneuploid genotype after prolonged passaging, the H7 and H9 cell lines were grown as previously described (Draper et al., 2004b) and compared after different culture passages (FIGS. 1-2).

After 87 passages, H7 cells showed gain of chromosome 1 and 12, consistent with previous reports. The majority of these late passage H7 cells were characterized by hyperploidy of specific chromosomes, suggesting clonal expansion of aneuploid cells in vitro. Only 10% of cells were euploid following quantification (FIG. 1). After 78 passages, H9 showed gain of chr 12 in 75% of cells.

By comparison, over 75% of early passage cells expanded for only 43 passages were numerically euploid (FIG. 1). The remainder of the populations was aneuploid, but distinguishable from the late passage H7 cells by consisting predominantly of hypoploid cells that were not obviously clonal (FIG. 1). This result suggested that distinct forms of chromosomal instability could occur following repeated passaging of HESCs, with markedly different influences on the euploid sub-population. Similar results were observed with all other HESCs examined.

To compare G banding and more detailed techniques for analyzing karyotypes, we used both G banding and SKY-PK to analyze two HESC lines (H7 and H9), which are 100% euploid at early passages. Comparatively low passage (H7 passage 43 (p43), H9 p37) and high passage cells (H7 p87, H9 p78) from each line were harvested using standard protocols (Barch, 1997). Samples were sent to an established cytogenetics lab for karyotyping via G banding. Samples from the same original split of hESCs were processed for SKY-PK in our lab. For each sample, forty metaphase spreads were analyzed by two independent observers. Individual karyotypes were documented in tabular format (Table I).

TABLE I

| Cell line | G banding | SKY-PK |
|---|---|---|
| H9p37 | 100% 46, XX | 72.5% 46, XX |
| | | 2.5% 40, XX, −2, −4, −7, +11, −14 |
| | | 2.5% 45, XX, −21 |
| | | 2.5% 46, XX, +3, −10 |
| | | 2.5% 43, XX, −10, −19, −21 |
| | | 2.5% 41, XX, −1, −3, −7, −16, −17 |
| | | 2.5% 46, XX, +5, −12 |
| | | 2.5% 44, XX, −5, −17 |
| | | 2.5% 45, XX, −20 |
| | | 2.5% 43, XX, −11, −15, −16 |
| | | 2.5% 42, XX, −13, −19, −20, −21 |
| | | 2.5% 45, XX, −19 |
| H9p78 | 78% 47, XX, +12 | 72.5% 47, XX, +12 |
| | 22% 46, XX | 20% 46, XX |
| | | 2.5% 48, XX, +6, +10 |
| | | 2.5% 50, XX, +12, +14, +15, +21 |
| | | 2.5% 47, XX, +1, −2, +12 |
| H7 p43 | 100% 46, XX | 80% 46, XX |
| | | 2.5% 39, X, −5, −11, −12, −14, −20, −22, −X |
| | | 2.5% 45, X |
| | | 2.5% 45, XX, −2 |
| | | 2.5% 42, XX, −14, −17, −20, −22 |
| | | 2.5% 43, XX, −17, −21, −22 |
| | | 2.5% 44, XX, −16, −22 |
| | | 2.5% 42, XX, −6, −11, −14, −17 |
| | | 2.5% 44, XX, −13, −21 |
| H7p87 | 90% 48, XX, +1, +12 | 77.5% 48, XX, +1, +12 |
| | 10% 46, XX | 10% 46, XX |
| | | 2.5% 47, XX, +1, −2, +12 |
| | | 2.5% 47 XX, +1, +12, −21 |
| | | 2.5% 47, XX, +1, −8, +12, |
| | | 5% 49, XX, +1, +11, +12 |

For early passage samples, G banding studies always reported a uniform rate of 100% euploid. In stark contrast, between 72.5-80% of hESCs were identified as euploid by SKY-PK. The early passage mosaic aneuploid population revealed by SKY-PK was non-clonal and largely but not exclusively hypoploid (see Table I for exact karyotypes). By contrast, G banding and SKY-PK produced similar although non-identical results for late passage hESCs, with the majority of cells showing clonal chromosome gains, along with a smaller, mosaic aneuploid component identified by SKY-PK.

The observed chromosome loss that accounts for most of the mosaicism is not due to technical artifact for several reasons: 1) a similar level of random chromosome loss is not seen in late passage hESC lines; 2) human lymphocytes, analyzed in parallel, were >97% euploid by SKY-PK, consistent with prior analyses (Rehen et al., 2001, 2005); 3) hyperploidies were also detected here and in previous studies. Moreover, our data from hESCs are consistent with the varying extent of aneuploidy observed in mouse ESCs (Eggan, 2002).

Chromosome Gain Correlates with Inhibition of Neuronal Differentiation

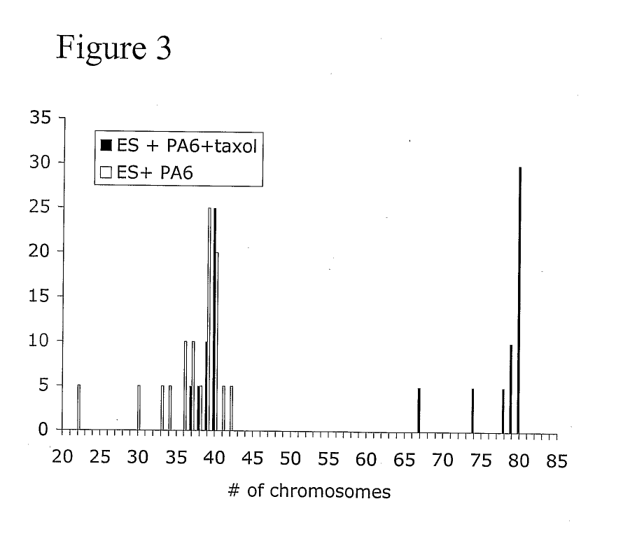

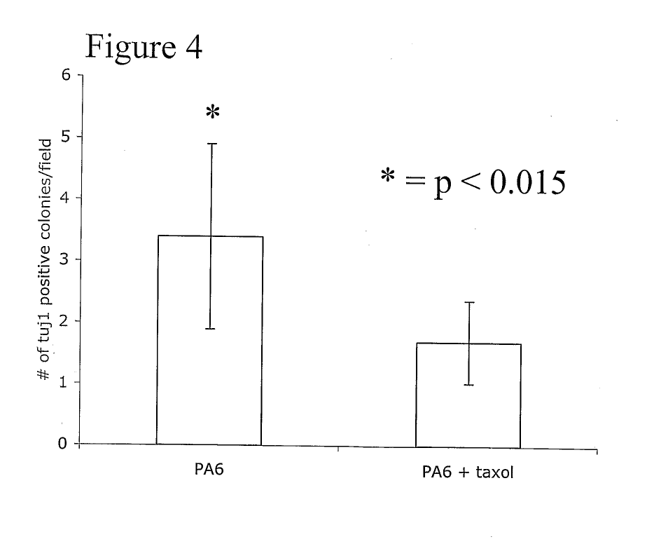

A key attribute of HESCs is their ultimate ability to differentiate into normal, mature cells. There is ample evidence that the differentiation of ES cells can be manipulated to give rise to enriched populations of neuronal cells. Stromal PA6 cells, when used as feeders, promote neural differentiation by inducing mouse ES colonies to become Tuj-1-positive and with robust neuritogenesis (Kawasaki et al., 2000). To examine the differentiation potential of anueploid vs. euploid cells, mammalian ESCs were treated with (aneuploid) or without (euploid) taxol and were examined for their ability to differentiate into neurons when cocultured with PA6 cells. Following 1 week of differentiation, 55% of taxol treated cells had gained chromosomes (FIG. 3). Moreover, when cells were fixed and stained for tuj1, there was a significant decrease in the number of neuronally differentiated colonies in the taxol treated cultures. These results show that different levels and forms of aneuploidy can alter the neuronal differentiation potential of cultured ESCs.

### Discussion:

A long-term goal of stem cell research is to develop new therapies for the treatment of debilitating diseases. In order to achieve this goal it will be necessary to obtain HESC lines that demonstrate reproducible properties even after extended passaging. The existence of chromosomal instability in HESC lines could alter the physiological properties of HESCs. Using SKY combined with quantification of the forms of aneuploidy in HESCs, pervasive aneuploidy of many distinct forms was observed. The functional consequences of aneuploidy in mammalian ESCs had not been previously examined, and surprisingly, at least two forms of aneuploidy—hyper vs. hypoploidy—are non-equivalent: hyperploidy but not hypoploidy correlates with inhibition of differentiation, at least along neuronal lineages.

The use of SKY to identify forms of aneuploidy has not been extensively reported previously on HESCs, contrasting with more classical cytogenetic techniques of past studies. SKY allows the unambiguous identification of both chromosome identity and translocations and has been used extensively in the study of cancer (Schrock et al., 1996; Difilippantonio et al., 2000). In addition to SKY, quantification of scores of metaphase spreads rather than a comparative few sample spreads (Amit et al., 2000; Buzzard et al., 2004; Draper et al., 2004b; Mitalipova et al., 2005) identified a surprisingly large and diverse range of chromosome gains and losses within HESCs and distinguished general differences between early and late passages (hypoploidy vs. hyperploidy).

These technical approaches revealed that chromosomal instability does not necessarily produce a conserved pattern of aneuploidy in HESCs. After 87 passages, 90% of the H7 cells were aneuploid, with the majority of cells showing chromosomal gains of chromosomes 1 and 12. The trend to gain chromosomes was observed in all late passage mammalian ESCs and particularly HESCs that we have examined, including HESCs from non-NIH sources.

What is the physiological significance or therapeutic risk associated with HESC aneuploidy? Aneuploidy—particularly hyperploidy—has been linked to cancer formation (Lengauer et al., 1997, 1998; Rajagopalan et al., 2003; Lengauer and Wang, 2004; Rajagopalan and Lengauer, 2004), which remains one possible risk factor associated with using HESCs. Other possibilities include karyotypic abnormalities that likely interfere with germline transmission of targeted mutations (Liu et al., 1997) and are a major cause of failure in obtaining contributions to all tissues of the adult mouse chimera (Longo et al., 1997). These latter results in mouse suggested that chromosomal instability, particularly hyperploidy and translocations, might lead to reductions in HESC pluripotentiality. Consistent with this view, hyperploid ESCs cells were unable to differentiate significantly along neuronal lineages.

By comparison, hypoploidy is compatible with normal levels of differentiation. It is reminiscent of normal developmental aneuploidy observed in mouse neuroprogenitor cells (Rehen et al., 2001; Kaushal et al., 2003; Yang et al., 2003; McConnell et al., 2004, Kingsbury et al., 2005) and mouse ES cells (Eggan et al., 2002). The presence of hypoploidy in neural cells has been shown to be compatible with normal differentiation in mouse and human neurons (Kaushal et al., 2003; Rehen et al., 2005). It remains possible that other phenotypic or functional differences exist in aneuploid neurons, however these differences may well represent what is observed in the normal nervous system (Kingsbury et al., 2005).

Overall, these results distinguish hyperploidies as an undesirable genotype for normal differentiation of mammalian ESCs, although such cells could have other advantageous properties By comparison, hypoploidy did not interfere with normal differentiation, and its existence appears to reflect what is normally observed in developing tissues. It is important to note that these distinctions were identified through the use of sensitive techniques like SKY, and quantification of chromosome loss and gain that is distinct from current standards of limited sampling used by most clinical cytogenetics laboratories. Recently it was shown that current NIH-funded HESC lines are contaminated with Neu5Gc, against which many humans have circulating antibodies (Martin et al., 2005). Non-physiological aneuploidy could represent another variable to consider in assessing the therapeutic usefulness of a HESC line. The introduction of routine, sensitive karyotyping for both current and new HESC lines will increase the likelihood of successfully using HESCs by defining desirable lines based on their chromosomal mosaicism for use in HESC research and therapies.

### Methods:

### Culture of HESC

The H7 HESCs (WiCell Research Institute, Inc., Madison, Wis.) were cultured on mitotically inactivated (mitomycin C treated) mouse embryonic fibroblasts (MEF, Specialty media, Phillipsburg, N.J.) in DMEM/F12 Glutamax (Gibco, Carlsbad, Calif.), 20% “KNOCKOUT” serum replacement (Gibco), 0.1 mM non-essential aminoacids (Gibco), 0.1 mM β-mercaptoethanol (Gibco), and 4 ng/mL FGF-2 (R&D systems, Minneapolis, Minn.). Colonies were passaged with colagenase/tripsin (Gibco) every 5-6 days. Cells were immunoreactive for undifferentiated markers including Oct4, SSEA-4, TRA-1-60, TRA-1-81. In addition, more than 90% of the colonies showed alkaline phosphatase activity. Cells were immuno-negative for the murine embryonic marker SSEA-1 and neural markers such as Nestin, a neural precursor marker; Tuj-1 and Map2 (a+b), immature neuronal markers; NeuN, mature neuronal marker; GFAP and s100-β, astrocyte markers and O4, GSTπ and RIP, oligodendrocyte markers.

### Neuronal Differentiation of ESC:

Mouse ESC were co-cultured with PA6 cells (Kawasaki et al., 2002) for 1 week under differentiation conditions (DMEM/F12 Glutamax (Gibco, Carlsbad, Calif.), 10% “KNOCKOUT” serum replacement (Gibco), 0.1 mM non-essential aminoacids (Gibco) and 0.1 mM β-mercaptoethanol (Gibco). Cells were treated with taxol (Sigma) at a concentration of 2.5 nM.

### Immunofluorescence Staining:

Immunofluorescence staining was performed as previously described (Gage et al., 1995), using α-β-tubulin-III (Tuj-1; 1/1000, Covance). Secondary antibodies were purchased from Jackson ImmunoResearch.

### Spectral Karyotyping (SKY) of HESC:

HESC Chromosome spreads were obtained by standard protocols (Barch et al., 1997; Rehen et al., 2001). 4′,6-diamidino-2-phenylindole (DAPI) (Sigma) and the SKY H-10 kit (Applied Spectral Imaging, Inc., Carlsbad, Calif.) were used according to the manufacturer's instructions.

### Data Analysis:

For each sample, 40 metaphase spreads were captured and analyzed by SKY and 70 metaphase spreads counterstained with DAPI were captured for aneuploidy quantification.

### REFERENCES CITED IN EXAMPLE

Amit M, Carpenter M K, Inokuma M S, Chiu C P, Harris C P, Waknitz M A, Itskovitz-Eldor J, Thomson J A (2000) Clonally derived human embryonic stem cell lines maintain pluripotency and proliferative potential for prolonged periods of culture. Dev Biol 227:271-278.
Barch M J, Knutsen T, Spurbeck J L (1997) The AGT Cytogenetics Laboratory Manual. Lippincott: Philadelphia.
Buzzard J J, Gough N M, Crook J M, Colman A (2004) Karyotype of human ES cells during extended culture. Nat Biotechnol 22:381-382; author reply 382.
Carpenter M K, Rosler E S, Fisk G J, Brandenberger R, Ares X, Miura T, Lucero M, Rao M S (2004) Properties of four human embryonic stem cell lines maintained in a feeder-free culture system. Dev Dyn 229:243-258.
Cheng L, Hammond H, Ye Z, Zhan X, Dravid G (2003) Human adult marrow cells support prolonged expansion of human embryonic stem cells in culture. Stem Cells 21:131-142.
Difilippantonio M J, Zhu J, Chen H T, Meffre E, Nussenzweig M C, Max E E, Ried T, Nussenzweig A (2000) DNA repair protein Ku80 suppresses chromosomal aberrations and malignant transformation. Nature 404:510-514.
Draper J S, Moore H D, Ruban L N, Gokhale P J, Andrews P W (2004a) Culture and characterization of human embryonic stem cells. Stem Cells Dev 13:325-336.
Draper J S, Smith K, Gokhale P, Moore H D, Maltby E, Johnson J, Meisner L, Zwaka T P, Thomson J A, Andrews P W (2004b) Recurrent gain of chromosomes 17q and 12 in cultured human embryonic stem cells. Nat Biotechnol 22:53-54.
Eggan K, Rode A, Jentsch I, Samuel C, Hennek T, Tintrup H, Zevnik B, Erwin J, Loring J, Jackson-Grusby L, Speicher M R, Kuehn R, Jaenisch R (2002) Male and female mice derived from the same embryonic stem cell clone by tetraploid embryo complementation. Nat Biotechnol 20:455-459.
Gage F H, Coates P W, Palmer T D, Kuhn H G, Fisher L J, Suhonen J O, Peterson D A, Suhr S T, Ray J (1995) Survival and differentiation of adult neuronal progenitor cells transplanted to the adult brain. Proc Natl Acad Sci USA 92:11879-11883.
Kaushal D, Contos J J, Treuner K, Yang A H, Kingsbury M A, Rehen S K, McConnell M J, Okabe M, Barlow C, Chun J (2003) Alteration of gene expression by chromosome loss in the postnatal mouse brain. J Neurosci 23:5599-5606.
Kawasaki H, Mizuseki K, Nishikawa S, Kaneko S, Kuwana Y, Nakanishi S, Nishikawa S I, Sasai Y (2000) Induction of midbrain dopaminergic neurons from ES cells by stromal cell-derived inducing activity. Neuron 28:31-40.
Kawasaki H, Suemori H, Mizuseki K, Watanabe K, Urano F, Ichinose H, Haruta M, Takahashi M, Yoshikawa K, Nishikawa S, Nakatsuji N, Sasai Y (2002) Generation of dopaminergic neurons and pigmented epithelia from primate ES cells by stromal cell-derived inducing activity. Proc Natl Acad Sci USA 99:1580-1585.
Kingsbury et al., (2005) Aneuploid neurons are functionally active and integrated into brain circuitry. Proc. Natl. Acad. Sci. USA in press.
Lakshmipathy U, Pelacho B, Sudo K, Linehan J L, Coucouvanis E, Kaufman D S, Verfaillie CM (2004) Efficient transfection of embryonic and adult stem cells. Stem Cells 22:531-543.
Lengauer C, Wang Z (2004) From spindle checkpoint to cancer. Nat Genet. 36:1144-1145.
Lengauer C, Kinzler K W, Vogelstein B (1997) Genetic instability in colorectal cancers. Nature 386:623-627.
Lengauer C, Kinzler K W, Vogelstein B (1998) Genetic instabilities in human cancers. Nature 396:643-649.
Liu X, Wu H, Loring J, Hormuzdi S, Disteche C M, Bornstein P, Jaenisch R (1997) Trisomy eight in ES cells is a common potential problem in gene targeting and interferes with germ line transmission. Dev Dyn 209:85-91.
Longo L, Bygrave A, Grosveld F G, Pandolfi P P (1997) The chromosome make-up of mouse embryonic stem cells is predictive of somatic and germ cell chimaerism. Transgenic Res 6:321-328.
Martin M J, Muotri A, Gage F H, Varki A (2005) Human embryonic stem cells express an immunogenic nonhuman sialic acid. Nature Medicine 11:228-232.
McConnell M J, Kaushal D, Yang A H, Kingsbury M A, Rehen S K, Treuner K, Helton R, Annas E G, Chun J, Barlow C (2004) Failed Clearance of Aneuploid Embryonic Neural Progenitor Cells Leads to Excess Aneuploidy in the Atm-Deficient But Not the Trp53-Deficient Adult Cerebral Cortex. J Neurosci 24:8090-8096.
Mitalipova M M, Rao R R, Hoyer D M, Johnson J A, Meisner L F, Jones K L, Dalton S, Stice S L (2005) Preserving the genetic integrity of human embryonic stem cells. Nat Biotechnol 23:19-20.
Pera M F (2004) Unnatural selection of cultured human ES cells? Nat Biotechnol 22:42-43.
Rajagopalan H, Lengauer C (2004) Aneuploidy and cancer. Nature 432:338-341.
Rajagopalan H, Nowak M A, Vogelstein B, Lengauer C (2003) The significance of unstable chromosomes in colorectal cancer. Nat Rev Cancer 3:695-701.
Rehen S K, McConnell M J, Kaushal D, Kingsbury M A, Yang A H, Chun J (2001) Chromosomal variation in neurons of the developing and adult mammalian nervous system. Proc Natl Acad Sci USA 98:13361-13366.
Rehen S K, Yung Y C, McCreight M P, Kaushal D, Yang A H, Almeida B S, Kingsbury M A, Cabral K M, McConnell M J, Anliker B, Fontanoz M, Chun J (2005) Constitutional aneuploidy in the normal human brain. J Neurosci 25:2176-2180.
Reubinoff B E, Pera M F, Fong C Y, Trounson A, Bongso A (2000) Embryonic stem cell lines from human blastocysts: somatic differentiation in vitro. Nat Biotechnol 18:399-404.
Rosler E S, Fisk G J, Ares X, Irving J, Miura T, Rao M S, Carpenter M K (2004) Long-term culture of human embryonic stem cells in feeder-free conditions. Dev Dyn 229:259-274.
Schrock E, du Manoir S, Veldman T, Schoell B, Wienberg J, Ferguson-Smith M A, Ning Y. Ledbetter D H, Bar-Am I, Soenksen D, Garini Y, Ried T (1996) Multicolor spectral karyotyping of human chromosomes. Science 273:494-497.
Thomson J A, Odorico J S (2000) Human embryonic stem cell and embryonic germ cell lines. Trends Biotechnol 18:53-57.
Thomson J A, Itskovitz-Eldor J, Shapiro S S, Waknitz M A, Swiergiel J J, Marshall V S, Jones J M (1998) Embryonic stem cell lines derived from human blastocysts. Science 282:1145-1147.
Xu C, Inokuma M S, Denham J, Golds K, Kundu P, Gold J D, Carpenter M K (2001) Feeder-free growth of undifferentiated human embryonic stem cells. Nat Biotechnol 19:971-974.
Yang A H, Kaushal D, Rehen S K, Kriedt K, Kingsbury M A, McConnell M J, Chun J (2003) Chromosome segregation defects contribute to aneuploidy in normal neural progenitor cells. J Neurosci 23:10454-10462.
Zeng X, Cai J, Chen J, Luo Y, You Z B, Fotter E, Wang Y, Harvey B, Miura T, Backman C, Chen G J, Rao M S, Freed W J (2004) Dopaminergic differentiation of human embryonic stem cells. Stem Cells 22:925-940.

The above example is provided to illustrate the invention but not to limit its scope. Other variants of the invention will be readily apparent to one of ordinary skill in the art and are encompassed by the appended claims. All publications, databases, Genbank sequences, patents, and patent applications cited herein are hereby incorporated by reference.

What is claimed is:

1. A method for detecting and defining the aneuploid mosaic status of a population of stem or progenitor cells, the method comprising,
detecting the presence of aneuploid mosaicism in the population, wherein at least 3 different karyotypes are detected amongst different cells in the population.

2. The method of claim 1, wherein at least 30 cells in the population are screened for aneuploidy.

3. The method of claim 2, wherein the aneuploidy of the cells is recorded.

4. The method of claim 1, wherein at least 5 different karyotypes are detected within different cells in the population.

5. The method of claim 1, comprising detecting cells with a net hypoploid mosaic karyotype, and selecting from the cells with a net hypoploid mosaic karyotype a stem or progenitor cell line that is hypoploid for at least a portion of a chromosome for differentiation, further propagation, or transplantation.

6. The method of claim 1, comprising detecting cells with a net hypoploid mosaic karyotype, and selecting from the cells with a net hypoploid mosaic karyotype a stem or progenitor cell line that is hypoploid for at least a portion of a chromosome for drug screening.

7. The method of claim 1, comprising detecting cells with a net hyperploid mosaic karyotype, and selecting from the cells with a net hyperploid mosaic karyotype a stem or progenitor cell line that is hyperploid for at least a portion of a chromosome for differentiation, further propagation, or transplantation.

8. The method of claim 1, comprising detecting cells with a net hyperploid mosaic karyotype, and selecting from the cells with a net hyperploid mosaic karyotype a stem or progenitor cell line that is hyperploid for at least a portion of a chromosome for drug screening.

9. The method of claim 1, further comprising passaging stem cells through at least one cycle of cell division prior to the detecting step.

10. The method of claim 1, wherein the karyotype of at least 80% of the cells in the cell population is determined.

11. The method of claim 1, wherein the detecting step comprises Fluorescent in situ hybridization (FISH) including multiplex FISH.

12. The method of claim 1, wherein the detecting step comprises spectral karyotyping (SKY).

13. The method of claim 1, wherein the detecting step comprises G banding, DAPI, or flow cytometry.

14. A method of screening for an agent that preferentially inhibits cells incapable of differentiation compared to cells capable of differentiation into a desired cell type, the method comprising,
contacting the agent to cells having a net hyperploid mosaic karyotype, wherein the cells are incapable of differentiation into the desired cell type; and
selecting an agent that inhibits propagation of the cells.

15. The method of claim 14, further comprising the steps of:
contacting the agent to cells having a hypoploid karyotype, wherein the cells are capable of differentiation into the desired cell type; and
selecting an agent that inhibits propagation of the hyperploid cells incapable of differentiation but does not significantly inhibit the propagation of the hypoploid cells capable of differentiation into the desired cell type.

16. A method for maintaining or improving a stem cell or progenitor cell population, the method comprising,
detecting the karyotype of at least one chromosome or a portion thereof in cells in the cell population; and
separating cells with a euploid or hypoploid karyotype at the at least one chromosome or portion thereof in the cell population from cells that are hyperploid at the at least one chromosome or portion thereof, thereby maintaining or improving the stem cell or progenitor cell population by removing hyperploid cells while maintaining euploid and hypoploid cells in the cell population.

17. The method of claim 16, wherein the detecting and separating steps are performed by fluorescence activated cell sorting (FACS).

18. The method of claim 16, wherein following the separating step, the cells with the euploid or hypoploid karyotype are made to differentiate.

19. The method of claim 16, wherein following the separating step, the cells with the euploid or hypoploid karyotype are propagated.

20. The method of claim 16, wherein following the separating step, the cells with the euploid or hypoploid karyotype are transplanted into an individual.
